(19) United States
(12) Patent Application Publication
(10) Pub. No.: US 2023/0263665 A1
(43) Pub. Date: Aug. 24, 2023

(54) LASER METHOD, DEVICE AND SYSTEM FOR TREATING RETINAL DETACHMENT

(71) Applicants: Centre For Eye Research Australia Limited, East Melbourne, Victoria (AU); The University of Melbourne, Victoria (AU); Heriot Eyecare Pty. Ltd., Melbourne, Victoria (AU)

(72) Inventors: Wilson J. Heriot, Balwyn North VIC (AU); Bang Viet Bui, Balwyn North VIC (AU); Andrew Bernard Metha, Balwyn North VIC (AU)

(73) Assignees: Centre for Eye Research Australia Limited, East Melbourne, Victoria (AU); The University of Melbourne, Victoria (AU); Heriot Eyecare Pty. Ltd., Melbourne, Victoria (AU)

(21) Appl. No.: 18/024,154

(22) PCT Filed: Sep. 2, 2021
(86) PCT No.: PCT/AU2021/051020

§ 371 Date: Mar. 1, 2023

(30) Foreign Application Priority Data
Sep. 2, 2020 (AU) 2020903130

(51) Int. Cl. A61F 9/008; A61F 9/007

(52) U.S. Cl. CPC A61F 9/00821; A61F 9/00727; A61F 2009/00863

(57) ABSTRACT

A method of integrating or fusing at least a part of a retina and at least one of a retinal pigmented epithelium (RPE) and choroid underlying the retina and the RPE is disclosed. The method comprises photodehydrating at least some proximal fluid separating one or more of the retina, the RPE and the underlying choroid, with photodehydrating laser light to thereby allow direct contact between the retina and at least one or more of the RPE and choroid. The method further comprises drying at least some of the proximal fluid with a gas flowing at a rate of up to 200 ml/min and photocoagulating with photocoagulating laser light to thereby integrate or fuse at least part of the retina with one or both of the RPE and choroid. Also provided are a device and a system for integrating or fusing these tissues.

### FIELD OF THE INVENTION

The present invention relates to a laser method, device and system for treating retinal detachment. More particularly, this invention relates to laser method, device and system for treating retinal detachment comprising at least one laser source and a flow of a gas.

### BACKGROUND TO THE INVENTION

Tissues sometimes detach from each other due to injury or other pathology. One example is retinal detachment, a disorder in which the retina peels away from its underlying layer of support tissue. Initial detachment may be localised, but without rapid treatment the entire retina may detach, leading to vision loss and blindness.

The role of a peripheral retinal tear in the causation of rhegmatogenous (from rhegma a discontinuity or break) retinal detachment (RRD) was proposed by Jules Gonin in 1904. Gonin subsequently developed the first successful technique for retinal detachment repair utilising a white-hot metal probe passed through a scleral incision. The thermal injury of the retina and adjacent retinal pigment epithelium (RPE) and choroid sometimes formed a watertight barrier between the subretinal space and the vitreous cavity resulting in a 30 to 40% cure rate. Thermal injury remains the basis for all retinal detachment repair, ranging from the historic hot metal probe to penetrating diathermy, and now contemporary cryo-retinopexy and laser treatment.

Retinal detachment (RD) causes blindness when retinal tears and “holes” allow vitreous fluid into the subretinal space, allowing the retina to float away from its proper RPE anchoring surface. Traditional retinal detachment repair utilises wound healing to create new (granulation) tissue to obliterate the subretinal space to seal the retinal tear margins. Laser or cryoretinopexy creates inflammation of the retina, RPE and underlying choroid. Scleral buckling or tamponade with gas or silicone oil, “clamps” the injured tissues together, while the wound matures over weeks and months to form a strong bond and seals the subretinal space access.

Traditional approaches to close the retinal tear using laser (photocoagulation), cryotherapy or diathermy can work poorly if the initial bond between the retina and the underlying RPE has not stabilised or matured before the withdrawal of the tamponade agent and allows fluid back under the retina. This can cause outright failure of the repair.

This means that there is a need for other procedures such as, leaving a much longer duration: gas, heavier-than-water liquid (perfluorocarbon) or silicon oil tamponade, inside the eye for many weeks to support the retina until the bond is strong enough.

In some cases, a second surgery is needed to remove the insoluble tamponade such as, perfluorocarbon liquid, silicone oil or a mixture of both.

In summary, with current approaches there is no immediate, waterproof seal formation around the retinal tear or hole to the underlying RPE such that there is a risk of (i) re-detachment; (ii) greater risk associated with complex procedures; and (iii) repeated procedures. In addition, air travel is prohibited while there is gas tamponade due to the intense intraocular pressure elevation at increasing altitudes that causes absolute blindness. An improved technique of retinal repair is desired.

International Patent Publication WO2014/110624, the publication of International Patent Application PCT/AU2014/0023 discloses a device for fusing tissue comprising a laser light source and/or a fluid. The fluid can be an air stream. In this publication, for the first time, Associate Professor Wilson Heriot disclosed his novel Retinal Thermofusion method in which the fluid between two tissues is eliminated by dehydration and the tissues are heated to fuse the tissues together.

U.S. Pat. Application Publication 2019/0343681, the publication of U.S. Pat. Application 16/270,996, and the co-pending Australian Patent Application Number 2019200894, progresses Associate Professor Wilson Heriot’s novel Retinal Thermofusion idea by providing some specific wavelengths for the laser.

U.S. Pat. Publication No. 2002/0082667 to Shadduck, teaches a surgical device for thermally-mediated treatments which uses a thermal energy delivery means to elevate the temperature of a biocompatible fluid media. The altered media may be a gas and has a high heat content and a high exit velocity. Different embodiments are described which have applications for endoscopic procedures. In the embodiment shown in FIG. 3, heating is performed with electrodes 40A, 40B and distally located electrical source 55. Paragraph [0046] describes heating the media to 100 to 400° C. and heating the tissue to a desired range of 65 to 100° C. very rapidly. As described in paragraph [0047] the heating is pulsed. While paragraph [0060] states that the device of D1 may be used for other anatomic structure or tissue volumes in endoscopic or open surgery, this is in the context of capturing and fusing or sealing tissue.

U.S. Pat. Publication No. 2009/0149846 to Hoey et al., teaches a device for applying a vapor source into a vaporisation chamber having a heating mechanism and applying energy from the heating mechanism to convert a substantially liquid media into a minimum water vapor level for causing an intended effect in tissue. In paragraph [0068] applications to ablation and thermotherapy and to treatment of a cornea and of a retina are described. Specifically, penetration through the sclera or cornea to treat retinal tissue, for example to ablate and coagulate blood vessels in the treatment of macular degeneration is described (paragraph [0070]). Paragraph [0070] describes an RF energy source 140 being operatively connected to a thermal energy source or emitter (e.g. opposing polarity electrodes 144a, 144b) in interior chamber 145. The heat of vaporisation is described as being in the range of 60 to 200° C. or 80 to 120° C. Suitable inflow pressure ranges are given as between 0.5 to 1,000 psi. Paragraph [0079] describes a resistive heating system. The heating mechanism can be either in the probe body 102 (FIG. 2) or located remotely (FIG. 6).

U.S. Pat. Publication No. 2005/0154384 to Benn-Nunn discloses a combination pressurised airflow and thermal cutting tool for use in eye cataract surgery. The handheld probe has an elongated, hollow body that has an air channel and is adapted to provide electrical power to a burning ring at a distal end.

U.S. Pat. Publication 2007/0239260 to Palanker et al. teaches a device for welding tissues to other tissues. The device described is quite simple, teaching adhering tissue at temperatures above 55° C. and below 100° C. (paragraph 5).

The paper titled “Diode-Pumped Tm:YAP Laser for Eye Microsurgery” to Jelínková et al., published in “Solid State Lasers XVII: Technology and Devices” edited by W. Andrew Clarkson, Norman Hodgson and Ramesh K. Shori in the Proceedings of Society of Photo-Optical Instrumentation Engineers (SPIE) Vol. 6871 details water absorption peaks for light for application in eye microsurgery.

The reference to any prior art in this specification is not, and should not be taken as, an acknowledgement or any form of suggestion that the prior art forms part of the common general knowledge.

### SUMMARY OF THE INVENTION

Generally, embodiments of the present invention relate to a laser method, device and system for treating retinal detachment.

In a broad form, the invention relates to a laser method, device and system for treating retinal detachment comprising at least one laser source and a flow of a gas.

In a first aspect, although it need not be the only or indeed the broadest aspect, the invention provides a method of integrating or fusing at least a part of a retina and at least one of a retinal pigmented epithelium (RPE) and choroid underlying the retina and the RPE, the method comprising:
photodehydrating at least some proximal fluid separating one or more of the retina, the RPE and the underlying choroid, with photodehydrating laser light to thereby allow direct contact between the retina and at least one or more of the RPE and choroid;
drying at least some of the proximal fluid separating the retina, the RPE and the choroid with a gas flowing at a rate of up to 200 ml/min; and
photocoagulating at least part of the retina and at least one of the RPE and the choroid with photocoagulating laser light to thereby integrate or fuse at least part of the retina with one or both of the RPE and choroid.

The method may also comprise determining tissue temperature. The tissue temperature may be determined by conducting spectral analysis.

In a second aspect, the invention provides a device for integrating or fusing at least a part of a retina and at least one of a retinal pigmented epithelium (RPE) and choroid underlying the retina and the RPE, the device comprising:
at least one source of laser light, the source of laser light providing photodehydrating laser light and photocoagulating laser light; and
at least one source of a gas;
a pump to impel the gas at a flow rate up to 200 ml/min.

In a third aspect, the invention provides a system for integrating or fusing at least a part of a retina with at least one of a retinal pigmented epithelium (RPE) and choroid underlying the retina and the RPE, the system comprising:
at least one source of laser light, the source of laser light providing photodehydrating laser light and photocoagulating laser light; and
at least one source of a gas;
a pump to impel the gas at a flow rate up to 200 ml/min;
a handpiece to direct the gas at or near the retina, RPE and/or choroid to be fused.

The device according to the second aspect or the system according to the third aspect may further comprise a console and/or one or more gas line connecting the pump and handpiece for delivery of the gas.

In one particular embodiment of any one of the above aspects, the photodehydrating laser light and/or the photocoagulating laser light is/are provided concurrently with the gas. In another particular embodiment, the gas flow is provided at a lower rate during photocoagulation than during photodehydration. In still another particular embodiment, gas flow is provided during photodehydration and no gas flow is provided during photocoagulation.

In one particular embodiment of any above aspect, the photodehydrating laser light and the photocoagulating laser light may be directed along a laser light path. The laser light path may comprise one or more optical fiber. In one embodiment, the one or more optical fiber comprises one optical fiber line for directing both the photodehydrating laser light and the photocoagulating light. In another embodiment, the one or more optical fiber line comprises a photodehydrating laser light optical fiber line connected to a photodehydrating laser light source and a photocoagulating laser light optical fiber line connected to a photocoagulating laser light source. The one or more optical fiber may be at least partially surrounded by cladding. The cladding may comprise a thickness of 100 to 200 µm.

According to any one of the above embodiments, the optical fiber may comprise a length of 1 to 5 metres. In one particular embodiment, the optical fiber may comprise a length of 2 metres. The optical fiber may comprise a blunt ended endoprobe.

According to any one of the above aspects, the photodehydrating laser light may comprise a wavelength of 950 to 3,500 nm; near infrared up to 5,500 nm; 1,389 to 1,500 nm; 1,900 to 2,000 nm; and/or 2,900 to 3,000 nm. In particular embodiments, the photodehydrating light comprises a wavelength of 1,470 nm or 1,940 nm. In one particular embodiment, the photodehydrating laser light comprises a wavelength of 1,940 nm.

In one embodiment of any one of the above aspects, the photocoagulating laser light may comprise a wavelength of 480 to 580 nm; or 760 to 860 nm. The photocoagulating laser light may comprise a wavelength for absorption by an endogenous biochemical such as, a pigment which may for example comprise, melanin or haemoglobin. In particular embodiments, the photocoagulating laser light may comprise a wavelength of 532 nm or 810 nm. In other embodiments, the photocoagulating light may comprise any clinically used wavelength to coagulate tissue such as, 577 nm (yellow), 595 nm (orange) 630 nm (red); 488 and/or 514.5 nm (argon blue-green), 514.5 nm (green); and/or 647 nm (krypton red). The photocoagulating laser light may comprise a wavelength of 480; 481; 482; 483; 484; 485; 486; 487; 488; 489; 490; 491; 492; 493; 494; 495; 496; 497; 498; 499; 500; 501; 502; 503; 504; 505; 506; 507; 508; 509; 510; 511; 512; 513; 514; 515; 516; 517; 518; 519; 520; 521; 522; 523; 524; 525; 526; 527; 528; 529; 530; 531; 532; 533; 534; 535; 536; 537; 538; 539; 540; 541; 542; 543; 544; 545; 546; 547; 548; 549; 550; 551; 552; 553; 554; 555; 556; 557; 558; 559; 560; 561; 562; 563; 564; 565; 566; 567; 568; 569; 570; 571; 572; 573; 574; 575; 576; 577; 578; 579; or 580. The photocoagulating laser light may comprise a wavelength of 760; 761; 762; 763; 764; 765; 766; 767; 768; 769; 770; 771; 772; 773; 774; 775; 776; 777; 778; 779; 780; 781; 782; 783; 784; 785; 786; 787; 788; 789; 790; 791; 792; 793; 794; 795; 796; 797; 798; 799; 800; 801; 802; 803; 804; 805; 806; 807; 808; 809; 810; 811; 812; 813; 814; 815; 816; 817; 818; 819; 820; 821; 822; 823; 824; 825; 826; 827; 828; 829; 830; 831; 832; 833; 834; 835; 836; 837; 838; 839; 840; 841; 842; 843; 844; 845; 846; 847; 848; 849; 850; 851; 852; 853; 854; 855; 856; 857; 858; 859; or 860 nm.

The photodehydrating light may comprise a wavelength greater than 900 nm. The photocoagulating laser light may comprise a wavelength less than 900 nm.

In another particular embodiment, the photocoagulating light may comprise a wavelength of 1,470 nm or 1,940 nm wherein the photocoagulating light is provided at an increased power relative to the photodehydrating light.

According to any one of the above aspects, the photodehydrating light may be provided at 5 to 120 mW; 10 to 100 mW; or 40 to 80 mW. In a particular embodiment, the photodehydrating light is provided at less than 90 mW.

In another embodiment of any one of the above aspects, the photocoagulating light may be provided at 90 to 200 mW; 120 to 180 mW; 150 to 170 mW; or at greater than 90 mW. The photodehydrating light may be provided at least 60; 70; 80; 90; 100; 110; 120; 130; 140; 150 mW lower than the photocoagulating light.

Advantageously, the photodehydrating light at 1,470 nm and/or 1,940 nm increases measured retinal adhesion to underlying tissue.

According to any one of the above aspects, the photodehydrating light is provided at a photodehydrating power which is less than the photocoagulating power of the photocoagulating light.

In one embodiment of any one of the above aspects, the photodehydrating laser light and the photocoagulating laser light may comprise an output power of 1 to 250 mW; or 50 to 180 mW. The output power may be controllable in increments of 10 mW. The output power may be calibrated onboard before delivery. The onboard calibration may comprise onboard monitoring.

The photocoagulation may comprise a change in state of opposing surfaces of two or more of the retina, RPE and choroid, so that two or more of the retina, RPE and choroid are joined and form a bond when returned to normal temperature.

In a particular embodiment, the laser beam may have a small footprint. The laser beam footprint may comprise a diameter of 100 µm to 1,000 µm.

According to any one of the above aspects, an aiming beam may be provided. The aiming beam may comprise a visible colour such as red or green. The aiming beam may comprise a standard output power. The aiming beam may comprise an adjustable visible brightness.

According to any one of the above aspects, the gas may comprise room air, an onboard tank or a medical gas supply system. The gas may be atmospheric air or may comprise one or more components of air, one particular embodiment being nitrogen. In one particular embodiment of any above aspect, the gas may comprise sterile air. The gas may be suitably “dry” or “desiccated” gas to dry or desiccate said targeted area. In one particular embodiment, the gas comprises a relative humidity of 50 to 60%. The gas may be filtered.

According to any one of the above aspects, the gas flow may comprise a flow rate of 1 to 200 ml/min; 5 to 150 ml/min; 10 to 135 ml/min; or 15 to 125 ml/min. The flow rate may comprise 1; 2; 3; 4; 5; 6 ;7; 8; 9; 10; 15; 20; 25; 30; 35; 40; 45; 50; 55; 60; 65; 70; 75; 80; 85; 90; 95; 100; 105; 110; 115; 120; 125; 130; 135; 140; 145; 150; 155; 160; 165; 170; 175; 180; 185; 190; 195 or 200 ml/min. The flow may comprise up to 200 ml/min; or up to 150 ml/min. In one particular embodiment the flow rate is 10 to 25 ml/min.

In a particular embodiment, the flow rate may comprise a photodehydration flow rate and a photocoagulation flow rate. The photodehydration flow rate may comprise a flow rate of 1 to 200 ml/min; 5 to 150 ml/min; 10 to 135 ml/min; or 15 to 125 ml/min. The photodehydration flow rate may comprise 1; 2; 3; 4; 5; 6 ;7; 8; 9; 10; 15; 20; 25; 30; 35; 40; 45; 50; 55; 60; 65; 70; 75; 80; 85; 90; 95; 100; 105; 110; 115; 120; 125; 130; 135; 140; 145; 150; 155; 160; 165; 170; 175; 180; 185; 190; 195; or 200 ml/min. The photodehydration flow may comprise up to 200 ml/minute or up to 150 ml/minute. In one particular embodiment, the dehydration flow rate is 10 to 25 ml/min. The photocoagulation flow rate may comprise 0 to 200 ml/min; 0 to 100 ml/min; or 0 to 75 ml/min. In a particular embodiment, the flow during photocoagulation may comprise 0 to 50 ml/min.

In one embodiment of any one of the above aspects, the pump may comprise a low flow pump.

According to any one of the above aspects, the pump may further comprise a regulator.

According to any one of the above aspects, a display may be comprised. The display may show one or more of: a current flow rate; a current wavelength; laser power output; laser pulse duration; laser repeat interval cycle; and/or laser pulse count.

According to the second or third aspect, the handpiece may comprise a probe. The handpiece may comprise a gas flow channel. The handpiece may comprise a 23G probe. The probe may comprise a 100 µm core. The probe may comprise a 600 µm circumference and/or a 515 µm inner circumference. The probe may comprise a wall thickness of 85 µm.

In another embodiment of the second or third aspect, the handpiece may comprise a 25G probe. The 25G needle may comprise a thin walled probe. The handpiece may comprise a 527 µm outer circumference and/or a 290 µm inner circumference. The probe may comprise a wall thickness of 119 µm.

In another embodiment of the second or third aspect, the handpiece may comprise a 27G probe. The 27G needle may comprise a thin walled probe. The handpiece may comprise a 413 µm outer circumference and/or a 210 µm inner circumference. The probe may comprise a wall thickness of 102 µm.

According to the second or third aspects, the handpiece may comprise a control to regulate the gas flow. The control may comprise one or more aperture. The regulation of the gas flow may be in 5 ml/min increments or flow on or off.

According to the second or third aspects, the gas flow may comprise a 10% variance.

According to any one of the above aspects, the gas flow may be calibrated by on-board monitoring before delivery.

According to the second or third aspect, the device or system may comprise tubing to conduct the gas. The tubing may comprise a length of 1 to 5 metres. In a particular embodiment, the tubing comprises a length of 2 metres. The tubing may comprise a diameter of 1 to 10 mm. In a particular embodiment, the tubing comprises a diameter of 3 mm. The tubing may comprise one or more connector for connection to a gas source. The one or more connector may comprise a standard or conventional connector. The tubing may comprise one or more filter at one or both ends. The one or more filter may comprise a syringe filter. The filter may comprise a 0.1 to 0.8 micron; 0.15 to 0.5 or 0.2 to 0.3 micron filter. In a particular embodiment the filter 0.2 micron filter.

According to any one of the above aspects, the proximal fluid may be within a diameter of 600 to 1,200 µm of a target area for integration or fusion. The proximal fluid may comprise sub-retinal fluid between the retina and the RPE that is to be eliminated or substantially eliminated.

The device or system of the second or third aspects may comprise a thermal imaging channel to allow spectral analysis to determine the tissue temperature. The thermal imaging channel may comprise a channel within the one or more optical fibers.

Further aspects and/or features of the present invention will become apparent from the following detailed description.

### BRIEF DESCRIPTION OF THE DRAWINGS

In order that the invention may be readily understood and put into practical effect, reference will now be made to embodiments of the present invention with reference to the accompanying drawings, wherein like reference numbers refer to identical elements. The drawings are provided by way of example only, wherein:

FIG. 1: shows a flowchart illustrating the steps according to one embodiment of a method according to the invention.

FIG. 2: is a diagram showing one embodiment of a device and system according to the invention.

FIG. 3A: is a schematic diagram showing another embodiment of a device according to the invention.

FIG. 3B: is a sectional view of the device of FIG. 3A.

FIG. 4A: is a bar graph showing porcine tissue peak temperature (°C) during laser treatment, using, from left to right: (i) a control at room temperature; (ii) a photocoagulation laser (532 nm) alone, i.e. with no drying; (iii) a 1,470 nm photodehydrating laser followed by 532 nm photocoagulation laser; (iv) a 1,940 nm dehydrating laser followed by a 532 nm photocoagulation laser; and both photodehydration and photocoagulation at 1,940 nm. Ns no significance.

FIG. 4B: is another bar graph showing measured horizontal force (gm) required to detach the untreated retina from the treated retina, using, from left to right: (i) a control at room temperature; (ii) a photocoagulation laser (532 nm) alone, i.e. with no drying; (iii) a 1,470 nm photodehydrating laser followed by 532 nm photocoagulation laser; (iv) a 1,940 nm dehydrating laser followed by a 532 nm photocoagulation laser; and both photodehydration and photocoagulation at 1,940 nm.

FIG. 5: is a table showing the data used for FIG. 4B. This data is repeated in Table 1.

FIG. 6A: is an OCT (Optical Coherence Tomography) image showing a baseline porcine retinal thickness.

FIG. 6B: is another OCT image showing a porcine retina after 3 minutes of drying using a 1,940 nm laser.

FIG. 6C: is a graph showing a plot of duration of retinal thermofusion drying (seconds) on the x-axis versus relative retinal thickness (%) on the y-axis, the 1,940 nm laser dehydration (open squares) thinned the retina significantly faster than warm air drying (open circles).

FIG. 7A; FIG. 7B; FIG. 7C: are photographs showing in vivo effects of 1,940 nm laser drying on the margins of induced retinal holes.

FIGS. 7D; 7E; and 7F: are photographs taken in vivo two weeks after surgery showing that the retina is attached and there is retinal choroidal bonding around the margins of the RTF repair site.

FIGS. 7G; 7H; 7I; 7J; 7K; and 7L; are further photographs showing that following tissue harvest and fixation, the eyecup shows that the margin of the retina is still adherent to the underlying choroid.

FIG. 8: FIG. 8A shows a retinal section stained with H&E (haematoxylin and eosin), highlighting a region that transitions (from right to left) from normal retina (see FIG. 8B), to detached retina, retinal scar tissue at the edge of the hole (see FIG. 8D), a region within the repaired hole where there is fusion of the RPE with the underlying choroid (see FIG. 8C).

FIG. 9: are images produced by thermodynamic modelling performed for the warm air emitting probe showing lateral air flow and heat spread (FIG. 9A); and significant elevation of intraocular temperature from heat radiating from the shaft (FIG. 9B).

FIG. 10: two images (FIG. 10A and FIG. 10B) show a 25 g laser spot size (footprint) on (FIG. 10A) the margin of a peripheral retinal tear in a human eye and (FIG. 10B) near the optic nerve showing the 25G laser probe, the optic nerve head (diameter of 1,550 µm for that patient) and the laser foot print (~200 µm) when compared to the internal reference size of diameter of 1,550 µm in that eye.

FIG. 11: screen capture images from a video showing: 1,470 nm laser light with no gas flow (FIG. 11A) and 1,470 nm laser light with gas flow (FIG. 11B) acting on water droplets; and graphs showing (FIG. 11C) that gas flow speeds water evaporation during photodehydration by 1,470 nm laser (FIG. 11C) and gas flow reduces surface tissue temperature during photodehydration (FIG. 11D). FIG. 11A: 1470 nm; 45 mW, 2.0 µL water drops; air flow at 5 ml/min. FIG. 11B: 1470 nm; 45 mW; 2.0 µL water drops; air flow at 20 ml/min.

Skilled addressees will appreciate that elements in the drawings are illustrated for simplicity and clarity and have not necessarily been drawn to scale. For example, the relative dimensions of some elements in the drawings may be distorted to help improve understanding of embodiments of the present invention.

### DETAILED DESCRIPTION OF THE INVENTION

The inventions relate to a laser method, device and system for treating retinal detachment.

The inventions are at least partly predicated on the unexpected discovery that a laser method, device and system comprising at least one laser light source and a gas flow is useful in treating retinal detachment.

A/Prof Wilson Heriot has provided the novel “retinal thermofusion” approach for treating retinal detachment. The rationale of this approach is that by removing all, most or at least some fluid from the subretinal space prior to laser photocoagulation, the retina and at least one of retinal pigmented epithelium (RPE) and underlying choroid will fuse into an integrated coagulum when heated with photocoagulation. This creates a waterproof seal immediately at the time of treatment thus preventing further fluid entry under the retina. Advantageously, this eliminates the need for postoperative tamponade with a gas or liquid. In addition, A/Prof Heriot’s method leads to a more effective “weld” or fusion giving any subsequent scarring process a head start. At present, without postoperative tamponade for weeks to months, a strong “weld” does not form and the retina may again detach, which is a known, inherent risk of the current treatments.

Advantageously, the inventors’ contribution increases confidence that only a single surgical procedure is needed. Furthermore, the strong initial repair should allow rapid postoperative return to normal life and reduce the chance of repair-failure, necessitating repeat surgery and further delay in recovery.

As will be elucidated below, in an in vivo rabbit model the inventors have shown that subretinal fluid photodehydration, using the drying laser wavelengths, with low gas flow, followed by photocoagulation or welding, using the coagulating laser wavelengths, in a single procedure, results in intraoperative attachment of the retina to the underlying tissue.

Although not wanting to be bound by any one theory, the inventors believe that the present invention evaporates at least some of the fluid wedge (meniscus and adjacent subretinal fluid) proximal to the tear(s) (i.e. the separated tissue) causing a retinal detachment using vaporising or evaporating light (photodehydration) and gas flow. By “proximal” is meant within a diameter of 600 to 1,200 µm for the tear or the tissue being targeted for integration or fusion. While not wanting to be bound by any one theory, the inventors hypothesise that to achieve the desired result, a specific aim within the drying of the proximal fluid is elimination or substantial elimination of sub-retinal fluid between the retina and RPE.

The inventors’ have discovered that, in one particular embodiment, lasers, or light, with wavelengths that are highly and specifically absorbed by water of endogenous fluid, without being absorbed by protein or pigment, dry the fluid in and under the retina. This step of drying sub-retinal fluid is important for improving the way that retinal detachments are repaired, by allowing an immediate strong initial bond to be made between the retina and the underlying retinal pigment epithelium (RPE).

Although not wanting to be bound by any one theory, the inventors hypothesis that photocoagulation may comprise a change in state of opposing surfaces of two or more of the retina, RPE and choroid, so that they are joined and form a bond when returned to normal temperature.

Advantageously, the footprint of the laser beam is small and contained, which has the benefit of significantly improved precision in drying and less damage to the surrounding tissue compared to methods that rely on gas flow alone. A small amount of gas flow, not enough to cause surrounding tissue dehydration, helps to move the water vaporised by the laser away from the wound, and has the additional advantage of preventing overheating of the tissue which could cause premature coagulation of one or both tissues and prevent effective fusion. Once the subretinal space is dried or partially dried, the detached retina can be fused (photocoagulation) to the underlying tissue (RPE and/or choroid) using: (a) laser light at a wavelength used for dehydration but with a higher power to coagulate using the tissue water as the energy absorbing agent; or (b) using laser light at a wavelength specific for photocoagulation. In one embodiment, the photocoagulation wavelength may be selected at a wavelength for absorption by an endogenous biochemical, for example, a pigment such as, melanin and/or hemoglobin.

In one particular embodiment, the purpose of the photodehydration step and/or the drying step is to remove sufficient fluid to allow photocoagulation to create an effective seal. It may not be necessary for all, or even a majority of the fluid to be removed. Sufficient fluid may be removed to allow contact between one or more of the retinal, RPE and underlying choroid.

In one embodiment, the inventors are the first to provide a console and/or handpiece, housing or providing two types of laser light: (i) a fluid drying, fluid vaporising or dehydrating laser light, that is “photodehydration” laser light and (ii) a photocoagulation laser light; and iii) a pump to deliver a continuous, and adjustable, stream of a gas.

Advantageously, the present invention provides for better patient outcomes (high benefit), requiring very little change to current practice (low risk).

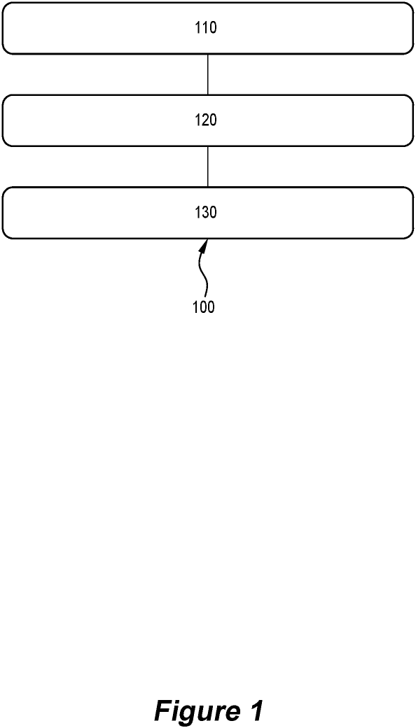

FIG. 1 shows one embodiment of a method 100 of fusing a retina and a retinal pigmented epithelium according to the invention. Method 100 comprises dehydrating 110 one or more of the retina, the retinal pigmented epithelium and the choroid underlying the retina and the RPE of at least some proximal subretinal fluid with a photodehydrating laser light, and drying 120 one or more of the retina, the retinal pigmented epithelium and at least some proximal subretinal fluid with gas flowing at a rate of up to 200 ml/min. The dehydrating laser light and gas may be provided concurrently, or step-wise. The photodehydration and/or drying to remove some proximal subretinal fluid allows direct contact between the tissues, i.e. between the retina and one or more of the RPE and the underlying choroid.

Method 100 also comprises photocoagulating or fusing 130 the retina with one or more of the retinal pigmented epithelium and choroid with photocoagulating laser light. The gas to dry may also be provided during the photocoagulating step 130 or no gas to dry may be provided during photocoagulating step 130.

The gas flow rate provided during the photodehydrating step 110 may be different to the gas flow rate during the photocoagulating step 130. To differentiate the two gas flow rates, the gas and gas flow during the photodehydrating step 110 may be referred to as photodehydration gas and photodehydration gas flow rate. Whereas the gas and gas flow, if any is provided, during the photocoagulation step 130 may be referred to as photocoagulation gas and photocoagulation gas flow rate.

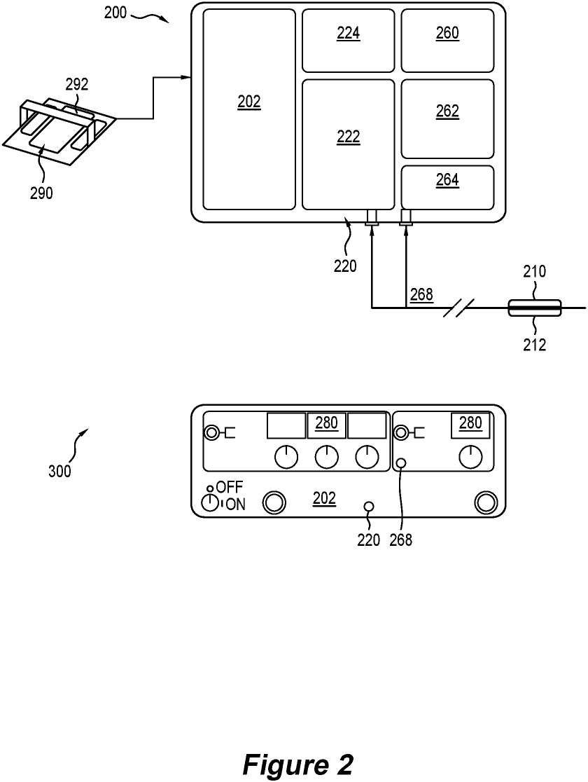

As shown in FIG. 2, the invention also provides a device 200 and system 300 for fusing a retina and a retinal pigmented epithelium comprising a body 202 housing a laser 220. The laser 220 comprises at least one laser source 222 providing photodehydrating laser light and photocoagulating laser light. Device 200 also comprises at least one source 260 of gas.

The inventors have surprisingly found that the gas must be provided at a low rate of up to, or not greater, than 200 ml/min to minimise adjacent tissue dehydration injury or elevation of the retinal tear edge. Observations in vivo during surgery on the rabbit eye, using a heated air handpiece, highlighted marked retinal surface drying and thinning in a penumbra beyond the direct line of airflow. This was reproduced in thermodynamic modelling (see FIG. 9 discussed below). The inventors have surprisingly demonstrated in vivo, in the rabbit eye, that gas flow above 200 ml/min would be unsuitable as this may lift retinal tissue when the airstream is at angle to drive gas under the retinal edge, and also cause a larger penumbra of dehydration in otherwise healthy retina.

To allow convenient control and adjustment of not only the gas flow but to also allow the laser activation and power output, device 200 comprises a foot control 290 which allows adjustment of these parameters by convenient, hands-free operation of one or more switch, pedal or button 292. For example, the power output of the laser 220 can be adjusted up or down by pressing an appropriate foot switch, pedal or button, up or down, respectively.

Device 200 and System 300 may also be provided with a remote-control unit 204 (not shown). The remote-control unit 204 may be operated from a position remote to the main console body 202, so that the main console body 202 is not contacted during use.

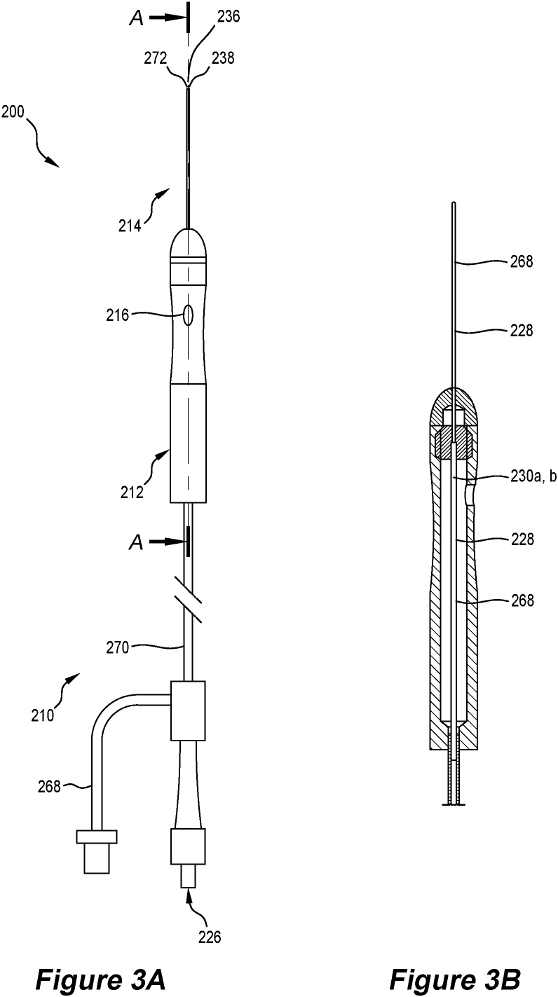

Device 200, shown in FIG. 2, is embodied as a console adapted to interface with handpiece 210 (see FIG. 3); foot control 290 and remote-control unit 204 (not shown). The handpiece 210 may be conveniently held for delivery and accurate direction of the laser light and gas flow for work inside the eye (intraocular). Handpiece 210 comprises body 212 on which is disposed a probe 214 for accurate direction of the laser light from laser outlet 236.

Handpiece 210 and probe 214 are sized to house: (i) optical fiber 228 to carry the light from laser source 222; and (ii) flexible tube 270 to conduct the gas flow from source of gas 260. In the embodiment shown, optical fiber 228 comprises a low-hydroxyl multimode optical fiber which provides better transmission for 1,940 nm light compared to standard optical fiber. The inventors have found such a low-hydroxyl multimode optical fiber to be advantageous when higher power is necessary for coagulation.

The embodiment shown also features the flexible tube 270 comprising a low-compliance flexible tube.

FIGS. 3A and 3B show one embodiment of handpiece 210 according to the invention. Handpiece 210 comprises a handpiece body 212 and probe 214.

The photodehydrating laser light and the photocoagulating laser light are directed along a laser light path 226 comprising one or more laser light optical fiber 228 which, in one embodiment, comprises one optical fiber line 230 for directing both the photodehydrating light and the photocoagulating light. In another embodiment, separate optical fiber lines 230 are provided, one line being a photodehydrating laser light optical fiber line 230a connected to a photodehydrating laser light source; and another separate line being a photocoagulating laser light optical fiber line 230b connected to a photocoagulating laser light source.

The one or more optical fiber 228 may be at least partially surrounded by cladding 232 (not shown). The cladding 232 may comprise a thickness of 50 to 200 µm. From the teaching herein, a skilled person can readily select appropriate cladding 232.

The one or more optical fiber 228 may comprise a length of 1 to 5 metres. In one particular embodiment, the one or more optical fiber 228 comprises a length of 2 metres. The one or more optical fiber 228 may comprise a blunt ended endoprobe.

The photodehydrating laser light may comprise a wavelength of 950 to 3,500 nm; near infrared up to 5,500 nm; 1,389 to 1,500 nm; 1,900 to 2,000 nm and/or 2,900 to 3,000 nm. In particular embodiments, the photodehydrating light comprises a wavelength of 1,470 nm or 1,940 nm. In one particular embodiment, the photodehydrating laser light comprises a wavelength of 1,940 nm.

The photocoagulating laser light may comprise a wavelength of 480 to 580 nm; or 760 to 860 nm. In particular embodiments, the photocoagulating laser light may comprise a wavelength of 532 nm or 810 nm. In other embodiments, the photocoagulating light may comprise any clinically used wavelength to coagulate tissue such as, 577 nm (yellow), 595 nm (orange) 630 nm (red); 488 and/or 514.5 nm (argon blue-green), 514.5 nm (green); and/or 647 nm (krypton red).

From the teaching herein, the skilled person will readily appreciate that each specific wavelength will have a different mode of action. For example, without wanting to be bound by any one theory, wavelengths of light that are at an absorption maxima for water, will photodehydrate fluid by energizing the inter-molecular bonds thus promoting vaporisation with only a mild elevation in tissue temperature.

In one particular embodiment, both the photodehydrating laser light and the photocoagulating laser light may be provided at a wavelength of 1,470 nm or 1,940 nm. When the photocoagulating light comprises a wavelength of 1,470 nm or 1,940 nm the photocoagulating laser light may be provided at an increased power relative to the dehydrating light power.

Advantageously, as is shown below, photodehydration with light at 1,470 nm and/or 1,940 nm increases measured retinal adhesion to underlying tissue. While not wanting to be bound by any one theory, the inventors have shown that while the photodehydration and drying appear to result in adhesion, that adhesion is reversed by rehydration as would occur in the eye while the photocoagulation following dehydration appears to be necessary to seal the tear irreversibly.

The tissue fusion into an integrated coagulum achieved with photocoagulation may be described as a type of “welding”. Studies detailed below show that a similar adhesion strength may be achieved with photodehydrating light at 1,940 nm followed by photocoagulating light also at 1,940 nm as compared to photodehydration with light at 1,940 nm and photocoagulation at 532 nm.

Again, without wanting to be bound any one theory, the mechanism of action of the photocoagulating wavelengths such as, 532 nm and 810 nm, may be explained by them being absorption maxima for pigment or other endogenous material, such as melanin and/or hemoglobin. That is, similar to the mechanism explained above with reference to the photodehydrating light, the photocoagulation may, at least in part, result from energizing the inter-molecular bonds of such an endogenous molecule or material, thereby promoting coagulation.

In particular embodiments, the photocoagulating laser light may comprise a wavelength of 480; 481; 482; 483; 484; 485; 486; 487; 488; 489; 490; 491; 492; 493; 494; 495; 496; 497; 498; 499; 500; 501; 502; 503; 504; 505; 506; 507; 508; 509; 510; 511; 512; 513; 514; 515; 516; 517; 518; 519; 520; 521; 522; 523; 524; 525; 526; 527; 528; 529; 530; 531; 532; 533; 534; 535; 536; 537; 538; 539; 540; 541; 542; 543; 544; 545; 546; 547; 548; 549; 550; 551; 552; 553; 554; 555; 556; 557; 558; 559; 560; 561; 562; 563; 564; 565; 566; 567; 568; 569; 570; 571; 572; 573; 574; 575; 576; 577; 578; 579; or 580.

In other particular embodiments, the photocoagulating laser light may comprise a wavelength of 760; 761; 762; 763; 764; 765; 766; 767; 768; 769; 770; 771; 772; 773; 774; 775; 776; 777; 778; 779; 780; 781; 782; 783; 784; 785; 786; 787; 788; 789; 790; 791; 792; 793; 794; 795; 796; 797; 798; 799; 800; 801; 802; 803; 804; 805; 806; 807; 808; 809; 810; 811; 812; 813; 814; 815; 816; 817; 818; 819; 820; 821; 822; 823; 824; 825; 826; 827; 828; 829; 830; 831; 832; 833; 834; 835; 836; 837; 838; 839; 840; 841; 842; 843; 844; 845; 846; 847; 848; 849; 850; 851; 852; 853; 854; 855; 856; 857; 858; 859; or 860 nm.

The photodehydrating light may comprise a wavelength greater than 900 nm. The photocoagulating laser light may comprise a wavelength less than 900 nm.

The photodehydrating light may be provided at 5 to 120 mW; 10 to 100 mW; or 40 to 80 mW. In a particular embodiment, the photodehydrating light is provided at less than 90 mW.

The photocoagulating light may be provided at 90 to 200 mW; 120 to 80 mW; or 150 to 170 mW; or at greater than 90 mW. The photodehydrating light may be provided at least 60; 70; 80; 90; 100; 110; 120; 130; 140; or 150 mW lower than the photocoagulating light.

Advantageously, the application of the photodehydrating light at 1,470 nm and/or 1,940 nm prior to coagulation increases measured retinal adhesion to underlying tissue. As noted above, the photocoagulation significantly strengthens the bond from photodehydration to a clinically useful amount to provide a clinically relevant seal to the tear because it is not reversible with rehydration.

In a particular embodiment, the laser beam may have a small footprint. The laser beam footprint is determined by the proximity of the probe tip to the retinal surface as controlled by the surgeon and by the size of the probe tip. Generally, the footprint range may comprise a diameter of 100 µm to 1,000 µm. The distance the probe is held from the target area is as per a convention clinical working distance, which may for example be 2 to 5 mm.

As used herein the term “footprint” when used in reference to the laser; laser beam; light; or laser light means the area of directly irradiated; illuminated or “lit”.

The photodehydrating laser light and the photocoagulating laser light may comprise an output power of 1 to 250 mW; or 50 to 180 mW. The output power may be controllable in increments of 10 mW. The output power may be calibrated onboard before delivery. The onboard calibration may comprise onboard monitoring.

Device 200 also comprises an aiming beam 224 which is used to provide a visible indicia such as, an area of coloured illumination. The colour may be red or green. Aiming beam 224 simplifies precise direction of the photodehydrating and photocoagulating laser light. Aiming beam 224 is inbuilt in laser 220 so that the aiming beam outlet 238 is the same as laser outlet 236. The aiming beam 224 may comprise a standard output power and may comprise an adjustable brightness.

The gas source 260 may comprise filtered room air, an onboard tank or a medical gas supply system. The gas may comprise sterile air, atmospheric air or may comprise one or more components of air such as, nitrogen or may comprise other suitable medical grade non-toxic gas. The gas may comprise sterile air. The gas may be suitably “dry” or “desiccated” gas to dry or desiccate the targeted area. To comply with operating room standards the gas may comprise a relative humidity of 50 to 60%.

The flow of gas and/or the flow of gas during photodehydration may comprise a flow rate of 1 to 200 ml/min; 5 to 150; 10 to 135; or 15 to 125 ml/min. The gas flow may comprise 1; 2; 3; 4; 5; 6; 7; 8; 9; 10; 15; 20; 25; 30; 35; 40; 45; 50; 55; 60; 65; 70; 75; 80; 85; 90; 95; 100; 105; 115; 120; 125; 130; 135; 140; 145; 150; 155; 160; 165; 170; 175; 180; 185; 190; 195 or 200 ml/min. The gas flow may comprise up to 200 ml/minute or up to 150 ml/minute. In one particular embodiment the gas flow is 10 to 25 ml/min. The gas flow during photocoagulation may comprise 0 to 50 ml/min.

As used herein by “flow of gas: is meant the flow exiting device 200. From the teaching herein, the skilled person will appreciate that any item in the gas line 268 impeding flow, such as a filter, may reduce the flow rate output from device 200.

The gas may be provided by a pump 262 which may comprise a low flow pump. In the embodiment shown in FIG. 2, pump 262 further comprises a regulator 264. The gas is provided through gas line 268 which comprises tubing or tube 270 to conduct the gas. The tube 270 may comprise a length of 1 to 5 metres and a diameter of 3 mm. The tube 270 comprises one or more connectors for connection to gas source 260. The one or more connector may comprise a standard or conventional connector. The tube 270 may comprise one or more filter at one or both ends such as, a syringe filter or a 0.2 micron filter. The gas line 268 delivers the gas to the gas outlet 272. In other embodiments, the filter may comprise a 0.1 to 0.8 micron (µm); 0.15 to 0.5 or 0.2 to 0.3 micron (µm) filter.

FIG. 2 also shows device 200 to comprise a display 280 which shows one or more of a current flow rate; a current wavelength; laser power output; laser pulse duration; laser repeat interval cycle; and/or laser pulse count.

In one embodiment handpiece 210 comprises a 23G aspirating probe comprising a 100 µm core; a 600 µm circumference; a 515 µm inner circumference; and a wall thickness of 85 µm.

In another embodiment, handpiece 210 comprises a 25G thin-walled probe comprising a 100 µm core; a 527 µm outer circumference; a 290 µm inner circumference; and a wall thickness of 119 µm.

In another embodiment handpiece 310 comprises a 27G thin-walled probe comprising a 100 µm core; a 413 µm outer circumference; a 210 µm inner circumference and a wall thickness of 102 µm. The 27G thin-walled probe may be from Hamilton Company USA.

From the teaching herein, the skilled person will appreciate that the gauge of the probe 214 and/or gas tube 270 may affect the gas flow rate. That is, a thinner gauge and/or tube 270 may lead to a faster jet pressure effect of the gas for the same volume delivered.

Handpiece 210 also comprises a control 216 to regulate the gas flow. The control 216 may comprise one or more aperture. Gas flow from the probe tip commences when the handpiece control 216 is closed by the surgeon’s finger. When open, the intraocular flow stops as the gas preferentially escapes thru the control 216 which offers dramatically less resistance than the narrow intraocular probe 214 irrespective of the pump settings. More subtle variation, with small increments in change of gas flow may be achieved with a control 216 comprising for example, a graduated slide. The regulation of the flow may also be controlled with more precision via foot control 290, in 5 ml/min increments. The gas flow may comprise a 10% variance. The gas flow may be calibrated by on-board monitoring before delivery.

The provision of control 216 is advantageous because it allows for instantaneous cessation of flow if there is a problem such as, retina lift.

Although not shown, the method 100 may also comprise determining tissue temperature. Device 200 may comprise a thermal imaging channel 234 (not shown) to allow spectral analysis to determine the tissue temperature. From the teaching herein, the skilled person will readily appreciate that visible changes may also be observed by the surgeon or other medical worker.

As will be shown below, advantageously, with an in vivo model of retinal detachment using living rabbit eyes, the inventors have shown that: both 1,470 nm and 1,940 nm lasers are effective at evaporating water or endogenous fluid; evaporation of water or endogenous fluid is faster if there is gentle gas flow to move the liberated water molecules away from the treatment site; gas flow, with a range of 1 to 200 ml/min, is suitable for this purpose; attachment of the retina to the underlying tissue can be achieved; and that the margin of the repair remains stable for the entire 2 week postoperative review period.

The inventors have determined that gas flow in the range of 1 to 200 ml/min is most suitable for this purpose.

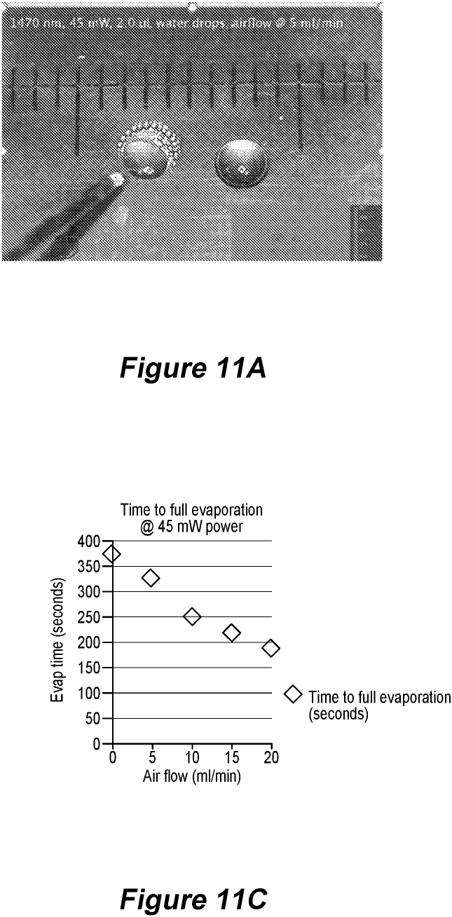

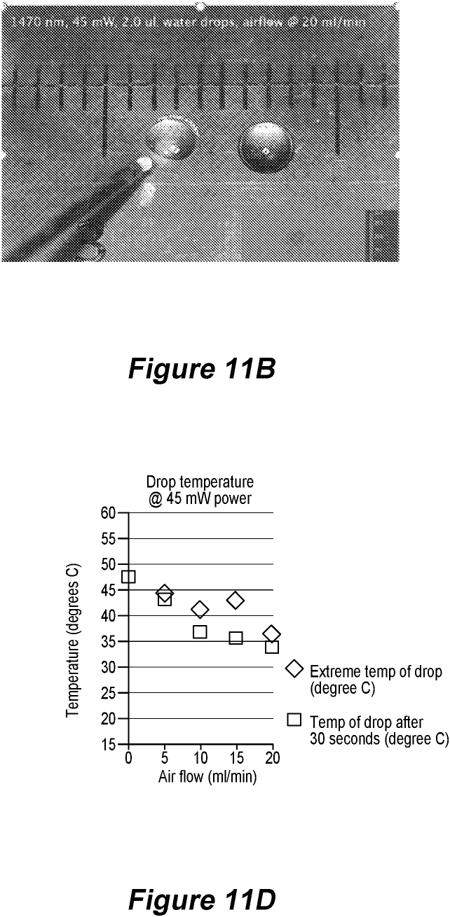

Using water droplets on slides, the inventors have shown that both 1,470 nm and 1,940 nm lasers are effective for evaporating water. The laser energy at these wavelengths is selectively absorbed by water thus energizing the inter-molecular bonds leading to liberation of water molecules as water vapor. The efficiency is greatly enhanced by the addition of low gas flow to disperse liberated water molecules (see FIG. 11 and discussion below). Efficient subretinal space dehydration occurs with some increased retinal temperature but not to coagulation levels within the photodehydration power range. The temperature elevation is noticeably less with the coaxial gas flow thus increasing the safety margin for dehydration without coagulation at the lower laser powers.

If during the photodehydrating step, additional power is provided, photocoagulation may occur, after an initial dehydration phase or may be initiated at the same time. The drying with the gas also provides some cooling, or reduction of temperature, which may lengthen the period of time until photocoagulation begins. To provide control over the transition between photodehydration and photocoagulation, the photodehydrating light and the photocoagulating light may be provided at two distinct power bands, with the photocoagulating light power higher than the photodehydrating light power. To promote photodehydration, the gas flow may be provided concurrently with the photodehydrating light. To promote photocoagulation, the gas flow may be provided at a lower flow than during the photodehydration or no gas flow may be provided.

Additionally, using models of retinal detachment of rabbit and porcine retinas, the inventors have shown that: 1,470 nm and 1,940 nm lasers are effective at evaporating water from the margin of the retinal tear; and laser drying of tissue is better with concurrent gentle gas flow.

Advantageously, the photodehydrating laser light of the invention may also be utilised with low flow or without any gas flow to coagulate tissue and/or bleeding sites instead of diathermy.

The following non-limiting examples illustrate the invention. These examples should not be construed as limiting: the examples are included for the purposes of illustration only. The Examples will be understood to represent an exemplification of the invention.

### EXAMPLES

### Major Activities

Measured temperature and adhesion strength of retinal detachment repair by RTF (Retinal Thermofusion) using 1,940 nm laser ex vivo on porcine eyes and compared it to previously tested warm air drying and 1,470 nm laser RTF.
Use of 1,940 nm laser for RTF in retinal detachment repair in vivo on rabbit eyes

### Specific Objectives

Testing of 1,940 nm laser for RTF ex vivo in porcine eyes and in vivo in rabbit eyes

### Results

### Measure the Adhesion Strength Immediately Following Laser and Hot Air Coagulation Fusion in ex Vivo Retina/RPE Models

Replacing the 1,470 nm laser with a 1,940 nm laser for drying as part of the RTF technique was investigated. The absorption peak of water molecules at 1,940 nm is approximately 5 times greater than 1,470 nm, so that the 1,940 nm laser diode liberates water molecules from hydrated tissue more effectively, resulting in faster dehydration of the subretinal water and the tissue. The 532 nm laser device was used for coagulation after drying with the 1,940 nm laser.

To validate the updated 1,940 nm laser module, the inventors performed ex vivo experiments with porcine eyes measuring the same parameters as for the 1,470 nm laser and included (see FIG. 5):
1) Porcine eye tissue temperature after applying 1,940 nm RTF;
2) Adhesion strength following retinal detachment repair by 1,940 nm RTF; and
3) Retinal thickness change by 1,940 nm laser RTF using optical coherence tomography.

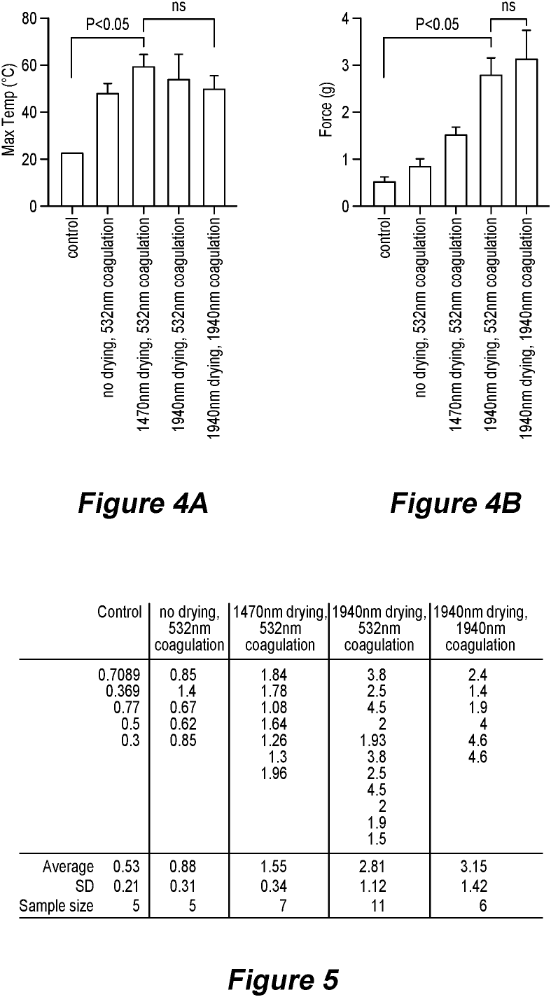

Tissue temperature and adhesion strength: The heat generated by the 1,940 nm laser was comparable to the 1,470 nm laser at similar dehydration power levels. There was no statistically significant difference. The average tissue temperature detected was approximately 55° C. (131° F.) as shown in FIG. 4A. This is an optimal temperature for effective drying without coagulating tissue. The measured force required to detach the retina after 1,940 nm RTF repair (2.81 - 3.15 gm) was significantly greater compared for the 1,470 nm laser (1.55 gm) and warm air drying (see FIG. 4B). The numerical data from these experiments is shown in FIG. 5 and Table 1 below.

The force measured in FIG. 4B is that to tear the untreated retina from the bonded area. In only a very small number of examples did the retina pull off Bruch’s membrane taking the RPE with it.

Speed of dehydration: the speed of tissue dehydration was assessed with optical coherence tomography to measure tissue thickness as a function of time following the onset of fusion.

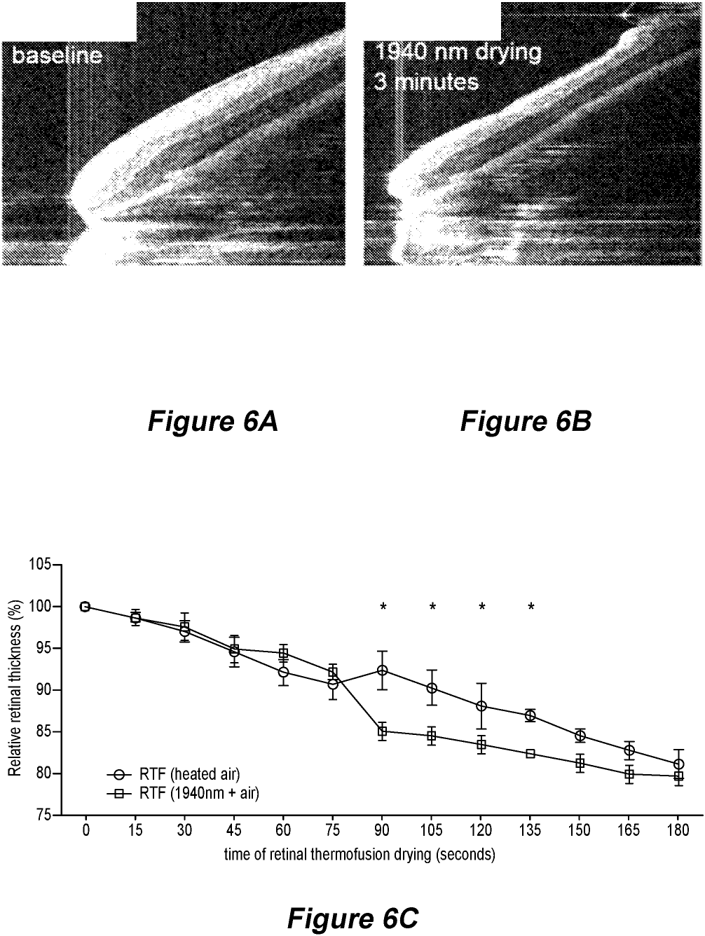

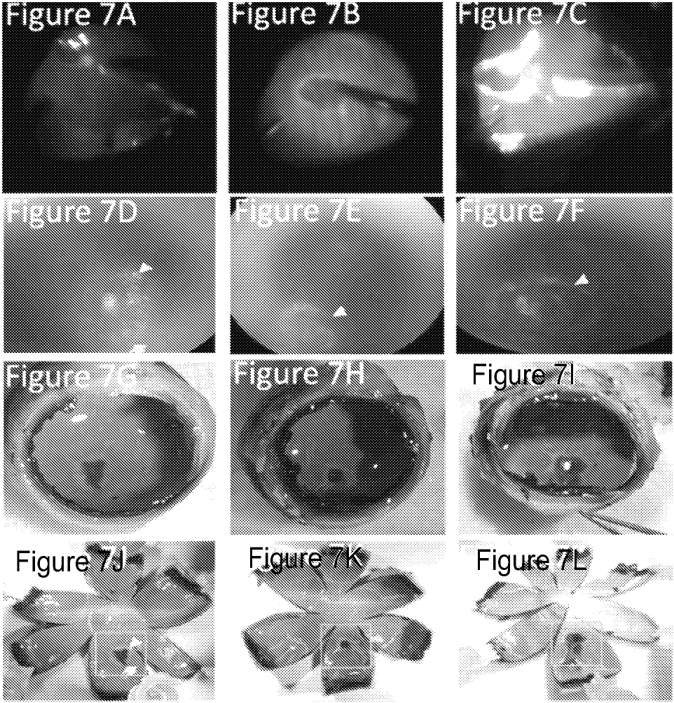

In FIGS. 6A, 6B and 6C, the warm-air drying method utilized in preliminary experiments (60 degree Celsius air at 100 ml/min flow rate), gradually thinned porcine retinal samples over the course of 3 minutes. At 3 minutes after drying onset, tissue had thinned to 83%. With RTF drying using the 1,940 nm with a cool air flow (50 ml/min) retinal samples had thinned to 85% after 90 seconds. There was significantly greater retinal thinning from 90 seconds onwards.

The longer wavelength (1,940 nm) laser produced a similar end point of dryness (p = 0.10) as the original approach employing heated air. Tissue drying time was reduced by 45 seconds (p <0.001), or by 40% of the time taken in the original approach.

Completed surgeries: the inventors have successfully completed 8 surgeries using the 1,940 nm laser for RTF repair of detached retina.

Retinal adhesion: FIG. 7 shows examples of the effect of the laser drying on the margins of the retinal hole in vivo. This can be seen as an increased reflectance of the retina (FIGS. 7A and 7C) as well as a slight whitening of the margin (FIG. 7B). Photographs taken in vivo two weeks after surgery show that the retina is attached and there is retinal choroidal bonding around the margins of the RTF repair site (FIGS. 7D, 7E and 7F). Following tissue harvest and fixation, the eyecup (FIGS. 7G, 7H, 7I and FIGS. 7J, 7K, 7L) shows that the margin of the retina is still adherent to the underlying choroid.

While the inventors do not want to be bound by any one theory, the scarring or wound healing appears to be an incidental inevitability of tear repair. The wound healing process will augment the sealing effect of the intraoperative fusion rather than diminish it and will follow the usual course of contemporary surgical technique where laser followed by tamponade creates an effective seal over time.

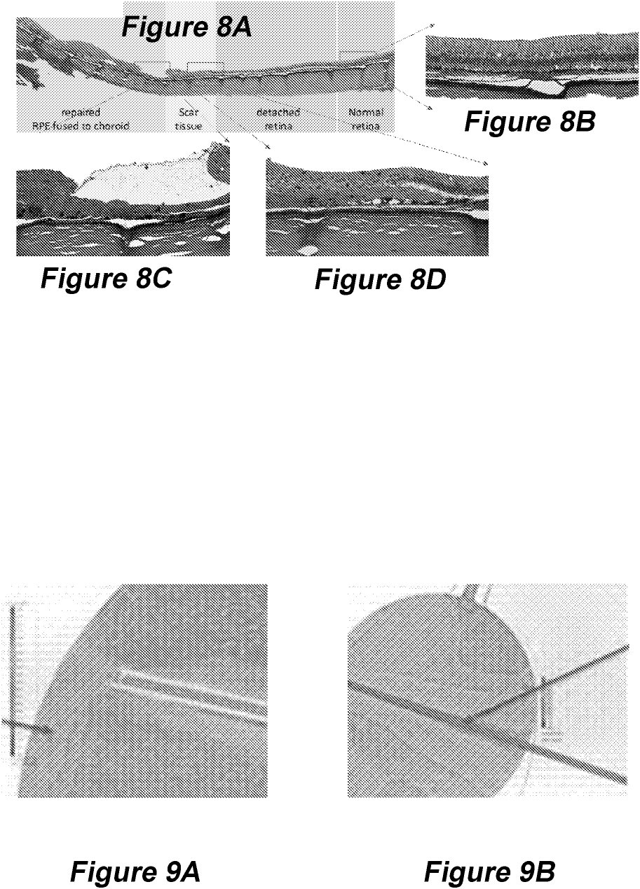

Histopathology of retinal adhesion: FIG. 8 demonstrates retinal adhesion two weeks after thermofusion repair using the 1,940 nm laser. Regions of normal retina, detached retina and retinal repair are evidenced in this cross section. These data provide robust evidence for the effectiveness of the RTF approach. FIG. 8C shows the edge of the repaired retinal break with reactive pigment epithelial proliferation. This may arise from hydraulic displacement of retinal pigment epithelial cells that occurred during creation of the retinal detachment.

Safety of 1,940 nm light during surgery: The backscatter of the 1,940 nm laser during treatment can be transmitted through the operating microscope to the surgeon and the assistant. To determine whether special protection from this invisible IR light is necessary, measurements were made with the source passing through a ZEISS OpMi operating microscope. The 1,940 nm laser unit was engaged, with the aiming beam (obligatorily) engaged to help alignment and set to deliver 20 mW at the tip. This output was measured by placing the tip at the entrance of an integrating sphere power meter (Thorlabs S148C - Integrating Sphere Photodiode Power Sensor, InGaAs, 1,200 to 2,500 nm, 1 W, 5 mm diameter aperture), ensuring that all light was collected. The tip was then set to point directly toward the microscope objective. The microscope height was adjusted so the fiber tip was clearly focussed, with magnification set to the lowest setting, to ensure maximum collection of light into the optical path. This configuration represents the “worse-case scenario” for the surgeon who actively observes the image of the irradiated retinal field - it is as if 100% of the light from the fiber tip underwent specular reflection and was re-directed towards the surgeon, and so this is an extremely conservative consideration.

At the lowest magnification, the microscope’s exit pupil position was 3 cm from the last eye-lens surface (for each ocular) and its diameter was 3 mm, falling entirely within the 5 mm aperture of the integrating sphere. The power measurements recorded for the 1,940 nm laser with a tip output of 20 mW, with the green filter (532 nm) in place ranged from 2 to 4 µW.

Measurements were made with and without the green 532 nm filter in place, and the results were negligibly different, with the final power measured at the exit pupil without the green filter ranged from 2 to 4 µW.

These findings confirm that the operating microscope itself already renders the 1,940 nm laser light safe for the surgeon and assistant. This is most likely because of the attenuation by the composition of the glass used in the microscope optics.

The amount of protection afforded is significant and sufficient, being at least a factor of 5,000, using conservative figures of 4 µW measured at the exit pupil from 20 mW directed upwards from the object field.

Thermodynamic modelling: FIGS. 9A and 9B showing thermodynamic modelling of intraocular warm air flow showing lateral air flow and heat spread (FIG. 9A); and significant elevation of intraocular temperature from heat radiating from the shaft (FIG. 9B), along with Table 2 (and Table 3) show the thermodynamic modelling performed for the warm air emitting probe. FIG. 9A highlights a lot of lateral air flow and heat spread. FIG. 9B shows significant elevation of intraocular temperature. This shows a wide area of retinal heating. As such the laser dehydration is dramatically better as a much lower airflow is needed. This minimizes both the area of dehydration to a very small asymmetric penumbra beyond the laser footprint and also minimises the risk of retinal tear edge elevation.

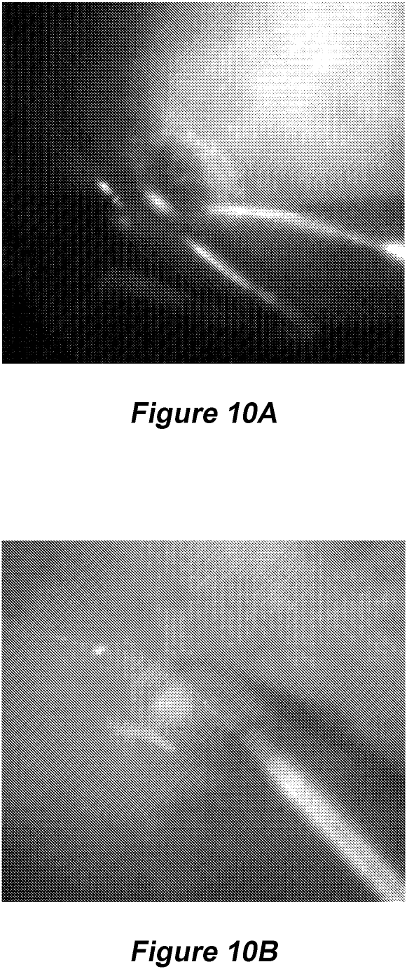

Human Eye Retinal laser for retinal tear: FIG. 10 shows a 25G laser probe and aiming beam (red) spot inside an air-filled human eye during vitrectomy. In FIG. 10A the incident aiming beam light spot is oval shaped because, as is the case here, most of the time, the probe cannot be oriented perpendicularly to the retinal surface. FIG. 10A also shows standard 532 nm laser whitish photocoagulation retinal burns surrounding the tear. The laser pulse width and power used was approximately 200 ms and 200 mW power. The right image, (FIG. 10B), shows a more perpendicular orientated aiming beam spot near the optic nerve showing that the spot size (footprint) is approximately 0.30 mm diameter judged relative to the optic nerve head as an internal reference (the optic nerve is approximately 1.500 to 1.650 mm in this subject.

From FIGS. 10A and 10B the skilled person will readily appreciate that the laser footprint is variable in shape and size, depending on tip-tissue distance and probe orientation. The inventors expect the size and shape of the infrared light distribution to be similar to the distribution of aiming beam light (red in this case). However, ultimately it is the size and shape of the coagulated zone (visualised in FIG. 10A as “whitish burns”) which may be used as a clinical guide during treatment application.

Photodehydration of water droplets: FIG. 11 shows laser photodehydration is enhanced by increasing gas flow to disperse liberated water molecules and that surface tissue temperature is diminished. FIGS. 11A and 11B are screenshots from video recording of 1,470 nm photodehydration of a 2 µL water droplet titrated onto a glass slide with a reference graticule underneath for scale. The laser HeNe aiming beam is reflecting red from the droplet (more obvious in FIG. 11B). A paired control droplet is on the right. FIG. 11A demonstrates significant dehydration of the illuminated droplet and the formation of micro-droplet condensation beyond the remaining main droplet. The adjacent control droplet remains unchanged during the experiment. FIG. 11B shows photodehydration of a 2 µL droplet with gas flow showing minimal condensation. FIG. 11C shows that under standard conditions, the time to full evaporation is related to the gas flow rate. FIG. 11D shows that surface temperature is lowered by the gas flow during photodehydration.

FIGS. 11A; 11B; 11C and 11D validate that the airflow is a significant factor enhancing photodehydration, and potentially, to a lesser extent, photocoagulation with light. Although an experiment with 1,940 nm laser light is not presented, from the teaching herein a skilled person will appreciate that the principle is the same.

In this specification, the terms “comprises”, “comprising” or similar terms are intended to mean a non-exclusive inclusion, such that an apparatus that comprises a list of elements does not include those elements solely, but may well include other elements not listed.

Throughout the specification the aim has been to describe the invention without limiting the invention to any one embodiment or specific collection of features. Persons skilled in the relevant art may realize variations from the specific embodiments that will nonetheless fall within the scope of the invention.

### TABLES

TABLE 1

Measured horizontal force (gm) required to detach the retina following treatment, using (i) a photocoagulation laser (532 nm) alone; (ii) a 1,470 photodehydrating laser followed by 532 nm photocoagulation laser; with a 1,940 nm drying laser followed by a 532 nm photocoagulation laser

| | Control | (i) | (ii) | (iii) | (iv) |
|---|---|---|---|---|---|
| | 0.7089 | 0.85 | 1.84 | 3.8 | 2.4 |
| | 0.369 | 1.4 | 1.78 | 2.5 | 1.4 |
| | 0.77 | 0.67 | 1.08 | 4.5 | 1.9 |
| | 0.5 | 0.62 | 1.64 | 2 | 4 |
| | 0.3 | 0.85 | 1.26 | 1.93 | 4.6 |
| | | | 1.3 | 3.8 | 4.6 |
| | | | 1.96 | 2.5 | |
| | | | | 4.5 | |
| | | | | 2 | |
| | | | | 1.9 | |
| | | | | 1.5 | |
| Average | 0.53 | 0.88 | 1.55 | 2.81 | 3.15 |
| SD | 0.21 | 0.31 | 0.34 | 1.12 | 1.42 |
| Sample size | 5 | 5 | 7 | 11 | 6 |

TABLE 2

Thermodynamic modelling

| Input/ Variables | Trial 1 | Trial 2 | Trial 3 | Trial 4 | Trial 5 | Trial 6 | Units |
|---|---|---|---|---|---|---|---|
| Needle Gauge# | 25 | 25 | 25 | 23 | 23 | 19 | # |
| Needle Inlet Temperature | 74.5 | 69.6 | 67.3 | 77.2 | 70.8 | 87.5 | °C |
| Target flowrate | | | | | | | |
| Target/seed flowrate | 0.05 | 0.1 | 0.2 | 0.05 | 0.1 | 0.05 | lpm |
| Quality of guess | 100% | 100% | 100% | 100% | 100% | 100% | |
| Iterate the “guess” in Target Flowrate until this value is close to 100% | | | | | | | |
| Target Needle Outlet Temperature | 65.0 | 65.0 | 65.0 | 65.0 | 65.0 | 65.0 | °C |
| Needle material | SS | SS | SS | SS | SS | SS | |
| Results | | | | | | | |
| Calculated Flowrate | 0.050 | 0.100 | 0.200 | 0.050 | 0.100 | 0.050 | l/min |
| Power Input | 53.55 | 96.39 | 183.93 | 56.02 | 99.04 | 66.41 | mW |
| Power Loss | 9.69 | 9.31 | 9.23 | 12.36 | 11.70 | 22.81 | mW |
| Basic Data | | | | | | | |
| Ambient pressure | 101325 | 101325 | 101325 | 101325 | 101325 | 101325 | Pa |
| Gas Constant | 287.06 | 287.06 | 287.06 | 287.06 | 287.06 | 287.06 | J/(kg.K) |
| air density (ambient) | 1.1959 | 1.1959 | 1.1959 | 1.1959 | 1.1959 | 1.1959 | kg/m3 |
| Specific Heat | 1019 | 1019 | 1019 | 1019 | 1019 | 1019 | J/kg.K |
| Needle length | 0.03 | 0.03 | 0.03 | 0.03 | 0.03 | 0.03 | m |
| Ambient Needle Surface | 37.8 | 37.8 | 37.8 | 37.8 | 37.8 | 37.8 | °C |
| Lab Ambient | 22.0 | 22.0 | 22.0 | 22.0 | 22.0 | 22.0 | °C |
| calculations | | | | | | | |
| Inner | 0.00026 | 0.00026 | 0.00026 | 0.000337 | 0.000337 | 0.000686 | m |
| Wall thickness | 0.000127 | 0.000127 | 0.000127 | 0.000152 4 | 0.000152 4 | 0.000191 | m |
| Inner Surface Area | 2.450E-05 | 2.450E-05 | 2.450E-05 | 3.176E-05 | 3.176E-05 | 6.465E-05 | m2 |
| Outside Surface Area | 4.844E-05 | 4.844E-05 | 4.844E-05 | 6.049E-05 | 6.049E-05 | 1.007E-04 | m2 |
| Section Area | 5.309E-08 | 5.309E-08 | 5.309E-08 | 8.920E-08 | 8.920E-08 | 3.696E-07 | m2 |
| Velocity | 15.70 | 31.39 | 62.78 | 9.34 | 18.69 | 2.25 | m/sec |
| Indicative pressure loss | 1178.49 | 4713.96 | 18855.82 | 417.54 | 1670.16 | 24.32 | Pa |
| Inner HTC | 33.77 | 37.23 | 40.70 | 31.17 | 34.64 | 24.06 | W/m2K |
| Wall conductivity | 16.100 | 16.100 | 16.100 | 16.100 | 16.100 | 16.100 | W/m.K |
| Outer HTC | 10 | 10 | 10 | 10 | 10 | 10 | W/m2K |
| Overall HTC | 6.307 | 6.531 | 6.730 | 6.207 | 6.452 | 6.071 | W/m2K |
| dT1 | 36.7 | 31.8 | 29.47275 601 | 39.36784 652 | 32.96219 333 | 49.7 | |
| dT2 | 27.2 | 27.2 | 27.2 | 27.2 | 27.2 | 27.2 | |
| LMTd | 31.713 | 29.440 | 28.321 | 32.910 | 29.989 | 37.327 | K |
| Heat Loss | 9.689E-03 | 9.315E-03 | 9.234E-03 | 1.236E-02 | 1.170E-02 | 2.281E-02 | Watt |
| Mass Flowrate Required | 1.001E-06 | 1.987E-06 | 3.987E-06 | 9.966E-07 | 1.993E-06 | 9.950E-07 | kg/s |
| Volumetric Flowrate Required | 8.370E-07 | 1.662E-06 | 3.334E-06 | 8.334E-07 | 1.667E-06 | 8.320E-07 | m3/sec |
| Target/Seed Flowrate | 8.333E-07 | 1.667E-06 | 3.333E-06 | 8.333E-07 | 1.667E-06 | 8.333E-07 | m3/s |
| Mass Flow | 9.966E-07 | 1.993E-06 | 3.986E-06 | 9.966E-07 | 1.993E-06 | 9.966E-07 | Kg/s |

TABLE 3

Needle Tables for calculations shown in Table 2

| Needle | | Nominal outer diameter | | | Nominal inner diameter | | | Nominal wall thickness | |
|---|---|---|---|---|---|---|---|---|---|
| Gauge | inches | Mm | tol. inches (mm) | inches | Mm | tol. inches (mm) | inches | mm | tol. inches (mm) |
| 7 | 0.18 | 4.572 | ±0.001 (±0.025) | 0.15 | 3.81 | ±0.003 (±0.076) | 0.015 | 0.381 | ±0.001 (±0.025) |
| 8 | 0.165 | 4.191 | ±0.001 (±0.025) | 0.135 | 3.429 | ±0.003 (±0.076) | 0.015 | 0.381 | ±0.001 (±0.025) |
| 9 | 0.148 | 3.759 | ±0.001 (±0.025) | 0.118 | 2.997 | ±0.003 (±0.076) | 0.015 | 0.381 | ±0.001 (±0.025) |
| 10 | 0.134 | 3.404 | ±0.001 (±0.025) | 0.106 | 2.692 | ±0.003 (±0.076) | 0.014 | 0.356 | ±0.001 (±0.025) |
| 11 | 0.12 | 3.048 | ±0.001 (±0.025) | 0.094 | 2.388 | ±0.003 (±0.076) | 0.013 | 0.33 | ±0.001 (±0.025) |
| 12 | 0.109 | 2.769 | ±0.001 (±0.025) | 0.085 | 2.159 | ±0.003 (±0.076) | 0.012 | 0.305 | ±0.001 (±0.025) |
| 13 | 0.095 | 2.413 | ±0.001 (±0.025) | 0.071 | 1.803 | ±0.003 (±0.076) | 0.012 | 0.305 | ±0.001 (±0.025) |
| 14 | 0.083 | 2.108 | ±0.001 (±0.025) | 0.063 | 1.6 | ±0.003 (±0.076) | 0.01 | 0.254 | ±0.001 (±0.025) |
| 15 | 0.072 | 1.829 | ±0.0005 (±0.013) | 0.054 | 1.372 | ±0.0015 (±0.038) | 0.009 | 0.229 | ±0.0005 (±0.013) |
| 16 | 0.065 | 1.651 | ±0.0005 (±0.013) | 0.047 | 1.194 | ±0.0015 (±0.038) | 0.009 | 0.229 | ±0.0005 (±0.013) |
| 17 | 0.058 | 1.473 | ±0.0005 (±0.013) | 0.042 | 1.067 | ±0.0015 (±0.038) | 0.008 | 0.203 | ±0.0005 (±0.013) |
| 18 | 0.05 | 1.27 | ±0.0005 (±0.013) | 0.033 | 0.838 | ±0.0015 (±0.038) | 0.0085 | 0.216 | ±0.0005 (±0.013) |
| 19 | 0.042 | 1.067 | ±0.0005 (±0.013) | 0.027 | 0.686 | ±0.0015 (±0.038) | 0.0075 | 0.191 | ±0.0005 (±0.013) |
| 20 | 0.03575 | 0.9081 | ±0.00025 (±0.0064) | 0.02375 | 0.603 | ±0.00075 (±0.019) | 0.006 | 0.1524 | ±0.00025 (±0.0064) |
| 21 | 0.03225 | 0.8192 | ±0.00025 (±0.0064) | 0.02025 | 0.514 | ±0.00075 (±0.019) | 0.006 | 0.1524 | ±0.00025 (±0.0064) |
| 22 | 0.02825 | 0.7176 | ±0.00025 (±0.0064) | 0.01625 | 0.413 | ±0.00075 (±0.019) | 0.006 | 0.1524 | ±0.00025 (±0.0064) |
| 22s | 0.02825 | 0.7176 | ±0.00025 (±0.0064) | 0.006 | 0.152 | ±0.00075 (±0.019) | 0.0111 | 0.2826 | ±0.00025 (±0.0064) |
| 23 | 0.02525 | 0.6414 | ±0.00025 (±0.0064) | 0.01325 | 0.337 | ±0.00075 (±0.019) | 0.006 | 0.1524 | ±0.00025 (±0.0064) |
| 24 | 0.02225 | 0.5652 | ±0.00025 (±0.0064) | 0.01225 | 0.311 | ±0.00075 (±0.019) | 0.005 | 0.127 | ±0.00025 (±0.0064) |
| 25 | 0.02025 | 0.5144 | ±0.00025 (±0.0064) | 0.01025 | 0.26 | ±0.00075 (±0.019) | 0.005 | 0.127 | ±0.00025 (±0.0064) |
| 26 | 0.01825 | 0.4636 | ±0.00025 (±0.0064) | 0.01025 | 0.26 | ±0.00075 (±0.019) | 0.004 | 0.1016 | ±0.00025 (±0.0064) |
| 27 | 0.01625 | 0.4128 | ±0.00025 (±0.0064) | 0.00825 | 0.21 | ±0.00075 (±0.019) | 0.004 | 0.1016 | ±0.00025 (±0.0064) |
| 28 | 0.01425 | 0.362 | ±0.00025 (±0.0064) | 0.00725 | 0.184 | ±0.00075 (±0.019) | 0.0035 | 0.0889 | ±0.00025 (±0.0064) |
| 29 | 0.01325 | 0.3366 | ±0.00025 (±0.0064) | 0.00725 | 0.184 | ±0.00075 (±0.019) | 0.003 | 0.0762 | ±0.00025 (±0.0064) |
| 30 | 0.01225 | 0.3112 | ±0.00025 (±0.0064) | 0.00625 | 0.159 | ±0.00075 (±0.019) | 0.003 | 0.0762 | ±0.00025 (±0.0064) |
| 31 | 0.01025 | 0.2604 | ±0.00025 (±0.0064) | 0.00525 | 0.133 | ±0.00075 (±0.019) | 0.0025 | 0.0635 | ±0.00025 (±0.0064) |
| 32 | 0.00925 | 0.235 | ±0.00025 (±0.0064) | 0.00425 | 0.108 | ±0.00075 (±0.019) | 0.0025 | 0.0635 | ±0.00025 (±0.0064) |
| 33 | 0.00825 | 0.2096 | ±0.00025 (±0.0064) | 0.00425 | 0.108 | ±0.00075 (±0.019) | 0.002 | 0.0508 | ±0.00025 (±0.0064) |
| 34 | 0.00725 | 0.1842 | ±0.00025 (±0.0064) | 0.00325 | 0.0826 | ±0.00075 (±0.019) | 0.002 | 0.0508 | ±0.00025 (±0.0064) |
| 26s | 0.01865 | 0.4737 | ±0.00025 (±0.0064) | 0.005 | 0.127 | ±0.00075 (±0.019) | 0.0068 | 0.1734 | ±0.00025 (±0.0064) |

1. A method of integrating or fusing at least a part of a retina and at least one of a retinal pigmented epithelium (RPE) and choroid underlying the retina and the RPE, the method comprising:
photodehydrating at least some proximal fluid separating one or more of the retina, the RPE and the underlying choroid, with photodehydrating laser light to thereby allow direct contact between the retina and at least one or more of the RPE and choroid;
drying at least some of the proximal fluid separating the retina, the RPE and the choroid with a gas flowing at a rate of up to 200 ml/min; and
photocoagulating at least part of the retina and at least one of the RPE and the choroid with photocoagulating laser light to thereby integrate or fuse at least part of the retina with one or both of the RPE and choroid.

2. The method of claim 1 further comprising determining tissue temperature, optionally by conducting spectral analysis.

3. A device for integrating or fusing at least a part of a retina and at least one of a retinal pigmented epithelium (RPE) and choroid underlying the retina and the RPE, the device comprising:
at least one source of laser light, the source of laser light providing photodehydrating laser light and photocoagulating laser light;
at least one source of a gas; and
a pump to impel the gas at a flow rate up to 200 ml/min.

4. A system for integrating or fusing at least a part of a retina with at least one of a retinal pigmented epithelium (RPE) and choroid underlying the retina and the RPE, the system comprising:
at least one source of laser light, the source of laser light providing photodehydrating laser light and photocoagulating laser light; and
at least one source of a gas;
a pump to impel the gas at a flow rate up to 200 ml/min; and
a handpiece to direct the gas at or near the retina, RPE and/or choroid to be fused.

5. The device according to claim 3, further comprising a console and/or one or more gas line connecting the pump and handpiece for delivery of the gas.

6. The device according to claim 3, wherein the photodehydrating laser light and/or the photocoagulating laser light is/are provided concurrently with the gas.

7. The device according to claim 3, wherein gas flow is provided at a lower rate during photocoagulation than during photodehydration.

8. The device according to claim 3, wherein gas flow is provided during photodehydration and no gas flow is provided during photocoagulation.

9. The device according to claim 3, wherein the photodehydrating laser light and the photocoagulating laser light are directed along a laser light path.

10. The device according to claim 9 wherein the laser light path may comprises one or more optical fiber.

11. The device according to claim 10 wherein the one or more optical fiber comprises one optical fiber line for directing both the photodehydrating laser light and the photocoagulating light.

12. The device according to claim 10 wherein the one or more optical fiber line comprises a photodehydrating laser light optical fiber line connected to a photodehydrating laser light source and a photocoagulating laser light optical fiber line connected to a photocoagulating laser light source.

13. The device according to claim 3, wherein the photodehydrating laser light comprises a wavelength of 950 to 3,500 nm; near infrared up to 5,500 nm; 1,389 to 1,500 nm; 1,900 to 2,000 nm; and/or 2,900 to 3,000 nm.

14. The device according to claim 3, wherein the photodehydrating light comprises a wavelength of 1,470 nm or 1,940 nm.

15. The device according to claim 3, wherein the photodehydrating laser light comprises a wavelength of 1,940 nm.

16. The device according to claim 3, wherein the photocoagulating laser light comprises a wavelength of 480 to 580 nm; or 760 to 860 nm.

17. The device according to claim 3, wherein the photocoagulating laser light comprises a wavelength for absorption by an endogenous biochemical such as, a pigment which may for example comprise, melanin or haemoglobin.

18. The device according to claim 3, wherein the photocoagulating laser light comprises a wavelength of 532 nm or 810 nm.

19. The device according to claim 3, wherein the photocoagulating light comprises any clinically used wavelength to coagulate tissue such as, 577 nm (yellow), 595 nm (orange) 630 nm (red); 488 and/or 514.5 nm (argon blue-green), 514.5 nm (green); and/or 647 nm (krypton red).

20. The device according to claim 3, wherein the laser comprises a small footprint such as, a diameter of 100 µm to 1,000 µm.

21. (canceled)

22. (canceled)
